(19) United States
(12) Patent Application Publication
(10) Pub. No.: US 2024/0145052 A1
(43) Pub. Date: May 2, 2024

(54) MEDICAL INFORMATION PROCESSING APPARATUS, MEDICAL INFORMATION PROCESSING SYSTEM, AND NON-TRANSITORY COMPUTER READABLE MEDIUM

(71) Applicant: CANON MEDICAL SYSTEMS CORPORATION, Otawara-shi (JP)

(72) Inventors: Hayato OKUMIYA, Nasushiobara (JP); Atsuko SUGIYAMA, Nasushiobara (JP); Keisuke HASHIMOTO, Nasushiobara (JP); Mariko SHIBATA, Nasushiobara (JP)

(73) Assignee: CANON MEDICAL SYSTEMS CORPORATION, Otawara-shi (JP)

(21) Appl. No.: 18/495,924

(22) Filed: Oct. 27, 2023

(30) Foreign Application Priority Data
Oct. 31, 2022 (JP) 2022-174616

(51) Int. Cl. G16H 10/65; A61B 5/00; A61B 5/16; G10L 17/00

(52) U.S. Cl. CPC G16H 10/65; A61B 5/165; A61B 5/4803; G10L 17/00; A61B 2503/20

(57) ABSTRACT

A medical information processing apparatus is a medical information processing apparatus that stores medical information of a patient and includes a processing circuitry and a memory. The processing circuitry performs voice recognition processing on a voice of a user, inputs the medical information based on a result of the voice recognition processing, acquires a mental state of the user by analyzing the voice of the user, and stores an analysis result of a mental state analysis section in the memory.

### CROSS REFERENCE TO THE RELATED APPLICATIONS

This application is based upon and claims the benefit of priority from the Japanese Patent Application No. 2022-174616, filed on Oct. 31, 2022, the entire contents of which are incorporated herein by reference.

### FIELD

Embodiments described in the present specification and drawings generally relate to a medical information processing apparatus, a medical information processing system, and a non-transitory computer readable program.

### BACKGROUND

In the medical industry, voice input assistance of clinical information is spreading. As a result, by inputting a part of work of a medical worker such as a doctor and a nurse by voice, input other than keyboard operation is efficiently realized, and work efficiency such as automatic shaping using an AI technology of information input by voice is improved. Furthermore, an AI technology for analyzing feelings from information of input sentences and voices is also in a state of being studied to a practical stage, and for example, technologies such as emotion analysis of text information (such as positive and negative words), emotion analysis of voices (such as intonation, magnitude, and speed), a combination thereof, and emotion analysis by facial expressions in an image acquired from a camera have been developed.

Medical institutions have started focusing on collection and analysis of information by questionnaires and the like, focusing on patient satisfaction and medical worker satisfaction. This is also caused by competition among medical institutions in rapidly changing medical care, an increase in the number of people changing jobs due to burnout syndrome of medical workers, health conditions, and the like, an unexpected and continuous increase in work due to epidemics, and the like.

Under such a situation, the medical worker is often very busy, and there is no time to sufficiently acquire information such as a questionnaire and an interview related to the medical worker satisfaction, stress, and the like. Therefore, there is a problem that the user side cannot grasp deep psychology and latent consciousness. In addition, there is a problem that even if the feeling by the voice input is acquired, information acquisition cannot be sufficiently realized due to the likeliness of diffusion of personal information of a patient, noise in the field, and the like.

### BRIEF DESCRIPTION OF DRAWINGS

FIG. 1 is a diagram schematically illustrating an information processing system according to an embodiment;

FIG. 2 is a flowchart illustrating an example of learning processing according to the embodiment;

FIG. 3 is a flowchart illustrating an example of learning processing according to an embodiment;

FIG. 4 is a flowchart illustrating an example of inference processing according to an embodiment;

FIG. 5 is a diagram illustrating an example of a result output of the inference processing according to the embodiment; and

FIG. 6 is a diagram schematically illustrating an example of the information processing system according to the embodiment.

### DETAILED DESCRIPTION

According to one embodiment, a medical information processing apparatus is a medical information processing apparatus that stores medical information of a patient and includes a processing circuitry and a memory. The processing circuitry performs voice recognition processing on a voice of a user, inputs the medical information based on a result of the voice recognition processing, acquires a mental state of the user by analyzing the voice of the user, and stores an analysis result of a mental state analysis section in a memory.

Hereinafter, embodiments are described with reference to the drawings.

### First Embodiment

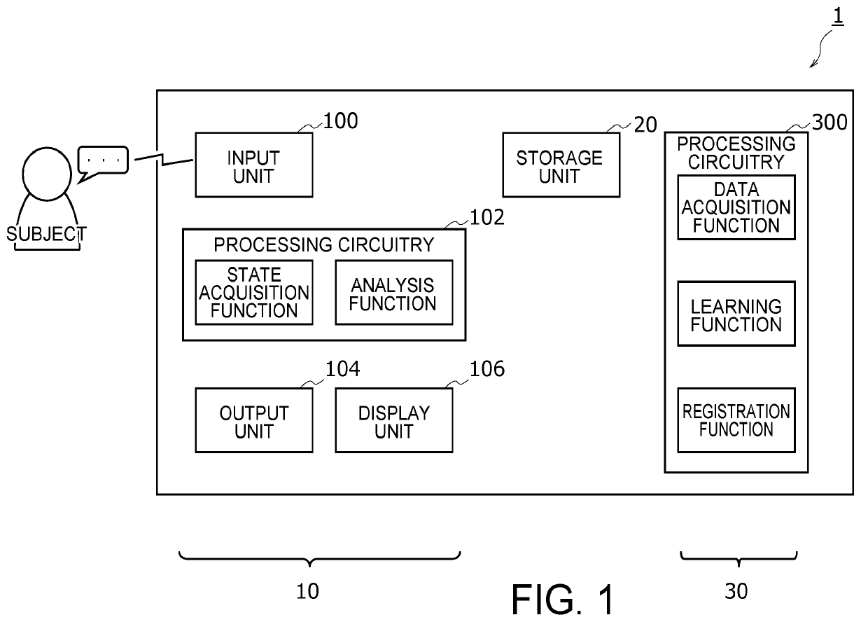

FIG. 1 is a diagram schematically illustrating an information processing system 1 according to an embodiment. As a non-limiting example, the information processing system 1 includes a first information processing device 10, a storage unit 20, and a second information processing device 30. Each of the first information processing device 10 and the second information processing device 30 can operate as a medical information processing apparatus.

The information processing system 1 is a system that acquires a voice uttered by a subject (hereinafter, a subject may be referred to as a user) who is a medical worker and infers a state such as a feeling, a fatigue level, and a stress level of the subject based on information of the voice.

As a non-limiting example, the first information processing device 10 includes an input unit 100 (input device), a processing circuitry 102, an output unit 104, and a display unit 106. The first information processing device 10 is an apparatus that performs an operation of acquiring data including voice information of the subject (first data) and an operation of outputting to a voice input by the subject (second data).

The first data is data for performing learning for inferring a state of a subject. The first data includes at least data obtained by inputting voice by the subject. The first data includes, for example, data including voice input data input for learning by the subject and information of an environment in which the voice is input or data including clinical information of a patient for which the subject performs voice input in daily work.

The second data is data acquired at the timing for acquiring the state of the subject and includes, for example, data including clinical information of a patient for whom a voice is input in daily work of the subject. In addition, the second data may be data obtained by acquiring a conversation other than the daily work of the subject as voice information. The second data is further used as data for inferring the state of the subject, and may be used as the first data for improving the accuracy of inference. By using the second data as the first data in this manner, the information processing system 1 can also improve the accuracy of learning by using more data.

The input unit 100 is an interface that acquires information for inferring a state of a subject. The input unit 100 includes, for example, a microphone that acquires voice information of the subject and acquires a voice of the subject. The input unit 100 acquires the first data and the second data. Furthermore, in a case of inputting a subject voice input environment or the like, the input unit 100 may include an arbitrary input device such as a keyboard, a mouse, or a touch panel.

The processing circuitry 102 (processor) is a circuitry that performs various kinds of processing in the first information processing device 10. In the present embodiment, the processing circuitry 102 has a state acquisition function of acquiring a state of a subject from voice information input by the subject (second data) and an analysis function of analyzing a threshold for determining that the state of the subject is a state different from the normal state. In the present embodiment, the state acquisition function configures a state acquisition unit, and the analysis function configures an analysis unit.

The state acquisition function compares the database for the voice information with the input second data to acquire the state of the subject. For example, the state acquisition function acquires information about the input second data and compares the acquired information with a threshold to determine whether the subject is in a normal state or a state different from the normal state. The information about the second data may be, for example, physical characteristics such as a pitch of voice, a tone, an utterance speed, or a magnitude (intensity) of voice. By determining these pieces of information for each subject, the state acquisition function acquires a state for each subject. This function is described in detail below with reference to an example.

Furthermore, the state acquisition function can also operate as a voice recognition processing function for inputting medical information by performing voice recognition processing on the voice of the user, and a mental state analysis function for obtaining the mental state of the user by analyzing the voice of the user. That is, the medical information processing apparatus can include a voice recognition processing unit (voice information processing section) and a mental state analysis unit (mental state analysis section) by this function. When the processing circuitry has the voice recognition processing function and the mental state analysis function, the medical information processing apparatus may configure the voice recognition processing unit and the mental state analysis unit.

The analysis function analyzes a temporal tendency of a state determined not to be a normal state in the state of the subject acquired by the state acquisition function. The analysis function analyzes, for example, a temporal state of the subject such as a stress check or a tendency of a temporal state change of the subject. In addition, the above-described mental state analysis function may operate as a part of the analysis function.

The output unit 104 outputs the state of the subject acquired by the processing circuitry 102. For example, the output unit 104 outputs information to the outside by displaying the state of the subject on the display unit 106. In addition, the output unit 104 may transmit the information to the outside by a message unit (not illustrated) in a system that transmits a message such as an e-mail or may store data in a circuit that stores data such as the storage unit 20.

Each of these functions may be stored in a storage circuit (not illustrated) in the first information processing device 10 in the form of a program. The processing circuitry 102 reads a program or an execution file from the storage circuit and executes the program or the execution file, thereby specifically realizing an information processing function by software corresponding to each program by using hardware resources. In other words, the processing circuitry 102 in a state read by each program has each function illustrated in the processing circuitry. Note that each processing function may be realized by a single processor, or the processing functions may be realized by combining a plurality of processors. Furthermore, the program may be stored not in the storage circuit of the first information processing device 10 but in an external storage circuit, or the program may be stored in a storage circuit arranged in a distributed manner.

In this manner, the first information processing device 10 acquires the state of the subject from the input second data and outputs the state of the subject.

The storage unit 20 includes a storage circuit that stores data. In FIG. 1, the storage unit 20 is in an independent form in the information processing system 1 but is not limited thereto. The storage circuit that forms the storage unit 20 may be independently provided as a file server in the information processing system 1 or may be formed by a plurality of storage circuits distributed on a cloud, for example. A part or all of the storage circuit may be provided inside the first information processing device 10 or may be provided inside the second information processing device 30. As described above, the storage unit 20 may be provided in a form accessible from an appropriate device of the information processing system 1 via a bus or a wired or wireless communication system. The storage unit 20 may include a mental state storage unit (mental state storage section) that stores the analysis result of the mental state analysis function. Here, the storage unit 20 is representatively described, but as described above, a storage circuit is provided in a plurality of devices, and these storage circuits can be collectively referred to as the storage unit 20. Furthermore, at least a part of the storage unit 20 may be arranged outside the information processing system 1.

The storage unit 20 stores a database learned and generated by the second information processing device 30 as at least a part of the function thereof, and the first information processing device 10 can refer to this database. In addition, the storage unit 20 may store the first data (including a case where the second data is used as the first data) acquired by the first information processing device 10. Furthermore, the storage unit 20 may store a program, an execution file, or the like for realizing information processing by software in some or all of the functions of the processing circuitry of the first information processing device 10 or the second information processing device 30 as at least some of the functions thereof.

The second information processing device 30 includes, for example, a processing circuitry 300. The second information processing device 30 creates a database for the first information processing device 10 to acquire the state of the subject from the second data.

The processing circuitry 300 (processor) is a circuitry that performs various types of processing in the second information processing device 30. In the present embodiment, the processing circuitry 300 has a data acquisition function, a learning function, and a registration function. In the present embodiment, the data acquisition function configures a data acquisition unit, the learning function configures a learning unit, and the registration function configures a registration unit.

The data acquisition function acquires first data including a combination of a state of a subject who is a medical worker and voice information. For example, with respect to the first data, the data acquisition function of the second information processing device 30 directly or indirectly acquires the data acquired by the first information processing device 10 via the input unit 100. The data acquisition function may also acquire information of a database to be learned from the storage unit 20.

The learning function extracts a parameter for acquiring a state of a subject from voice information of the subject based on the acquired first data and performs learning for classifying the state of the subject based on the extracted parameter. For example, the learning function generates a database for acquiring the state of each subject from voice data based on the first data acquired by the data acquisition function.

In the generation of the database, information of physical characteristics such as a pitch of a voice, a tone, an utterance speed, a waveform of a voice, or a magnitude (intensity) of a voice included in the voice data described above is acquired by analyzing the voice data included in the first data, and information such as a range and a combination of parameters in which the subject is in a normal state is learned based on the plurality of items of the first data. A general method can be used for acquiring the information such as the pitch of the voice from the voice data. The learning function can acquire the above parameters, for example, by performing rule-based processing or processing using a learned model on the voice data. The learning is not limited to the above, and any statistical method such as regression analysis, multiple regression analysis, or covariance analysis can be used.

The first data can include a situation in which the subject inputs a voice. The learning function can also perform learning by correcting the voice data based on this situation. The first data may include, for example, at least one of a place where the subject inputs a voice, a time zone, an input amount of data, a device to which the voice is input, a special circumstance when input is performed, and information obtained by semantically analyzing input contents. That is, as the first data, the data of the voice of the user may be stored in association with at least one of a place where the user inputs the voice, a time zone, an input amount of the data, a device to which the voice is input, a special circumstance when input is performed, and information obtained by semantically analyzing input contents.

The special circumstance when a voice is input includes, for example, a case where another medical worker performs input on behalf of the medical worker. The information obtained by semantically analyzing input contents can include, for example, a medical care content, a disease state, a disease name, a symptom, a treatment content, a medication state, or a situation such as surgery or death of a patient. The learning function learns to be able to infer the state of the subject with higher accuracy by changing a threshold or the like or weighting a parameter based on the special circumstances and/or the specific content of the voice data acquired by semantic analysis.

Note that test data can be mixed with the first data. The test data may be obtained by causing a subject to input a voice in daily work or outside work and separately inputting information such as a state of the subject at the timing, for example, information on whether the subject is in a normal state, or a feeling at the timing. The learning function can improve the accuracy of the association between the parameter and the state in the database as the learning progresses.

The registration function registers and reflects the learning result of the learning function in the database of the storage unit 20.

In this manner, the second information processing device 30 can form a database for acquiring the state of the subject based on the acquired first data by learning. As described above, the second information processing device 30 creates a database for each individual subject and registers the database in the storage unit 20.

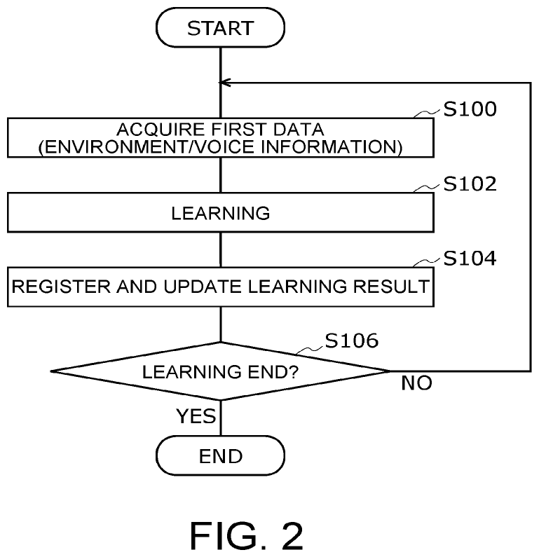

FIG. 2 is a flowchart illustrating an example of the learning processing according to the embodiment. This learning processing is mainly performed by the second information processing device 30 in FIG. 1. The input of the first data may be performed by the first information processing device 10. In the present embodiment, a mode of performing input for voice input learning as learning is described. This processing can also be used as generation of an initial determination criterion in an embodiment described below.

First, the first information processing device 10 acquires the voice data of the subject and, if necessary, data related to the environment (data of the place, the time zone, and the like described above) via the input unit 100 (S100). The first information processing device 10 may transfer the acquired first data to the second information processing device 30 or may store the acquired first data in the storage circuit such as the storage unit 20. The acquired first data is defined as, for example, data in a normal state.

The data acquisition function of the processing circuitry 300 of the second information processing device 30 acquires the first data acquired by the first information processing device 10, and the learning function performs learning for acquiring the state of the subject from the voice data of the subject described above (S102). In a case where the environment information is included in the first data, the learning function performs learning reflecting the environment information. As described above, the environment information includes, as non-limiting examples, an input place, an input time zone, an input amount, an input content, a terminal to be used, and other special conditions.

The input place can affect the volume of a voice, such as a noisy state in the surroundings, or a state in which the user has to speak in a small voice because the patient himself/herself or another patient is in front.

The input time zone may affect the speed of voice such as a relatively less busy time zone or a busy time zone such as immediately after the work shift. The input time zone may also affect the tone of voice related to work attitude, such as a state immediately after office attendance in the morning and a state of start/end of night duty.

The input amount may affect the speed and quality of voice in a case where the amount of data to be input, for example, the number of patients or the amount of clinical information on a per patient basis is extremely large or small. As another example, in a case where there is a deviation of a certain percentage or more with respect to the average number of patients input usually in one day, it is also possible to weight the condition.

In the input content, the content of the input such as the state of the patient, for example, a state in which the medical worker feels sad, the feeling of the subject performing input, for example, the stress state of the subject may affect the voice quality.

The terminal to be used is a factor related to a terminal such as voice recognition and terminal specifications and may affect voice quality.

The other state is, for example, proxy input, which is not a direct input of the clinical information of the patient in charge by the medical worker himself/herself, but is a special factor that the content of the input does not affect the feeling transfer due to operation of inputting the clinical information of another medical worker on behalf of the medical worker.

The factors caused by the respective above environments are given as examples, and in addition to these, various effects related to the voice can be exerted.

As an example, the learning function learns a combination of data and a threshold by an arbitrary statistical method so that a state of the subject can be appropriately determined from a voice by associating the environment information with each state of the voice or the like. Note that the above factors are not limited to being used alone, and a plurality of factors may be used in combination.

Furthermore, the learning function can also perform learning using a defined normal state. The defined normal state may be designated by the administrator or the individual subject or may be set from an average state of a predetermined number of times.

The first data may further include information indicating whether the subject is normal, together with the environment information and the voice data. In this case, the learning function can perform learning so as to appropriately determine whether the subject is normal based on the above factors and voice data.

As a result, in the processing of S102, the learning function can determine, based on the input environment of the subject, which voice indicates that the state of the subject is a normal state, and which voice indicates that the state of the subject is not a normal state. The learning function learns a pattern of a normal voice input of each individual, and learns a determination criterion for determining an abnormality from a voice input different from a normal state or a sign of the voice input.

For example, the learning function generates, by learning, a determination criterion for determining whether a pitch of a voice, a tone, an utterance speed, a waveform of a voice, or a magnitude of a voice is equivalent to those in a normal state under the above environment. This determination criterion may be expressed by a function in consideration of the above factors or may be expressed by a combination of thresholds for each parameter (such as pitch of voice). It is also possible to set a threshold for each parameter to be determined and a combination of the respective parameters.

As a non-limiting example, the learning function generates, by learning, a criterion such that it can be determined that it is not in a normal state in a case where there is an abnormal portion in the combination of the parameters as compared with the combination of the thresholds (in a case where the parameter deviates from the range of the normal state).

By performing learning using the first data defined as in the normal state, the learning function can generate a criterion as to which parameter (or combination of parameters) is in which range in a normal state.

The registration function registers a learning result and performs update registration, if necessary (S104). This result may be stored in the storage unit 20.

The processing circuitry 300 determines whether learning is completed (S106) and repeatedly performs learning when learning is not sufficient (S106: NO). In this case, it is not essential to acquire the voice data again in the first information processing device 10, and learning for improving accuracy of the existing data may be performed.

When it is determined that the learning is sufficient (S106: YES), the learning is ended.

As described above, according to the present embodiment, it is possible to acquire the determination criterion indicating whether the input voice data is in a normal state or other states by learning using the voice input data in a normal state. Note that, by using data in a state other than a normal state for learning, it is possible to perform learning for realizing classification with higher accuracy as to whether it is a normal state.

In a case where a voice is input, it is possible to appropriately determine whether the subject is in a normal state or the subject is not in a normal state by performing determination using this criterion. For example, it is also possible to grasp a stress sign such as a state in which stress is excessively applied and prevent burnout. In addition, the sound state of the medical worker can be managed, and as a result, it can contribute to the quality and reliability of medical care in the facility.

### Second Embodiment

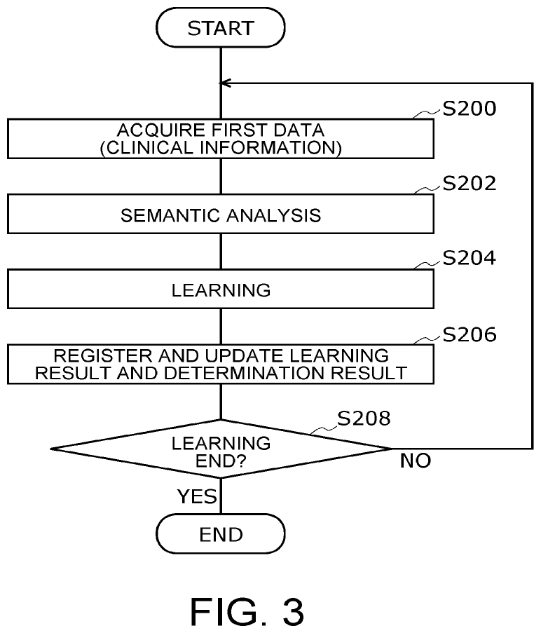

FIG. 3 is a flowchart illustrating an example of processing of a learning function according to an embodiment. In the present embodiment, an information processing system 1 that performs learning by using voice-input data of actual clinical information is described.

The data acquisition function acquires, via the first information processing device 10, data obtained by inputting clinical information by a voice as the first data (S200). In a case where the first information processing device 10 acquires the environment information, the data acquisition function can acquire the environment information together with the data obtained by inputting the clinical information by a voice.

The data acquisition function performs semantic analysis of the clinical information and acquires information related to the medical care of the patient included in the voice data of the first data (S202). This semantic analysis may use any semantic analysis method. In addition, any voice-text conversion method can be used for semantic analysis.

The learning function performs learning similar to that of the first embodiment (S204). However, unlike the first embodiment, the learning function can perform learning with reference to the content of the semantically analyzed clinical information.

Compared with the first embodiment, the learning function can further add, for example, the type of the input clinical information and the semantically analyzed content to the elements of the analysis. As a result, the learning function can realize analysis in consideration of the type of input clinical information or the influence of the semantically analyzed medical care content on the feeling of the medical worker. It is also possible to set an influence that gives a condition to each type and content of the clinical information.

The type of clinical information may be, for example, data acquired from a medical chart and an electronic medical chart instead of voice data such as a medical record, a surgical record, a death certificate, a birth certificate, and an instruction record. However, this does not exclude voice input of the medical chart itself.

As an example, in a case where the patient dies, the voice input of the subject may be determined to be normal, or there may be a tendency of the voice input that the voice is lower and slower than usual.

In a case where there is a claim from a patient, there may be a tendency of the voice input that a voice is stronger than usual.

In a case where a consultation is received from a patient, there may be a tendency of the voice input that a voice is higher than usual.

The above description is given as an example, and the present invention is not limited thereto. As described above, the learning function can also perform learning in consideration of a change in the voice input according to the medical care content (including a semantic analysis of the voice input).

By performing learning using the clinical information, the second information processing device 30 can detect a state different from the normal state at the time of inputting clinical information by voice in the daily work of the medical worker.

The learning function can further extract data in which the input voice is in a state different from the normal state to generate a result database. The registration function may register and update data in a state different from the normal state in the result database together with the learning result (S206). The result database may be stored, for example, in the storage unit 20.

The generated result database may be available for secondary use. The result database can be used, for example, by aggregating and referring to states of the department unit to which the subject belongs and the subject individual unit. By using this aggregation result, it is also possible to display an alert such as high stress on a per department basis or on a per individual basis or transmit an e-mail or a message.

As described above, according to the present embodiment, the information processing system 1 can also acquire voice data in daily work of a subject who is a medical worker. By learning the determination criterion based on voice data in daily work, it is possible to determine the state of the medical worker with higher accuracy. In addition, by generating the result database, it is also possible to perform a statistical stress check on a subject in any range such as for each department or for each individual.

### Third Embodiment

In the above description, a learning phase is mainly described, but in the present embodiment, an inference phase is mainly described.

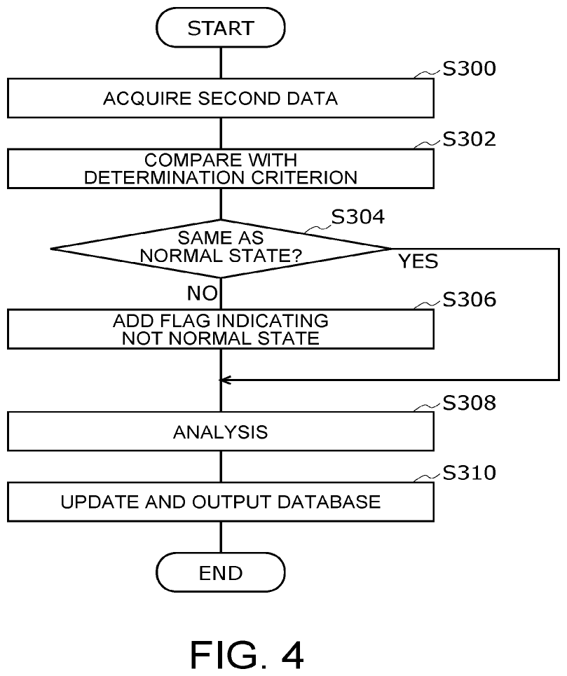

FIG. 4 is a flowchart illustrating an example of inference processing according to the embodiment. The inference is performed by the first information processing device 10 based on the criterion generated by the second information processing device 30.

The first information processing device 10 acquires the second data to be inferred via the input unit 100 (S300). The second data is, for example, voice data for inputting clinical information related to the daily work of the subject and may be used for learning as a part of the first data as described above.

The state acquisition function compares the second data with the determination criterion and performs comparison for determining whether the state of the subject who has input the second data is a normal state (S302). The state acquisition function compares the determination criterion learned by the second information processing device 30 described in each of the above embodiments with the second data.

The state acquisition function determines whether the determination result is the same state as that in the normal state from the comparison result (S304). In a case where the determination result is different from the normal state (S304: NO), the state acquisition function adds a flag indicating that the determination result is not the normal state to the determined second data (S306).

After the flag is added or when it is determined that the determination result is the normal state (S304: YES), the analysis function analyzes the temporal tendency of the registered result database and the second data to which the result flag determined by the state acquisition function is added.

The analysis function can infer that the stress state of the subject is not so bad, for example, in a case where it is determined that it is not a normal state sporadically in time series. On the other hand, in the time series, in a case where the determination result of the state that is not in a normal state gradually increases, or in a case where the determination result of the state not in the normal time suddenly increases, a tendency that a considerable stress accumulates in the subject is inferred.

The result analyzed by the analysis function may be reflected in, for example, the result database and may be output to an appropriate user such as a supervisor via the output unit 104 (S310). In addition, data that is determined to be not in a normal state by the state acquisition function can also be registered in the result database.

With this registration in the result database, the analysis function can analyze the temporal tendency of the state that is not a normal state by referring to the result database and the determination result of the second data.

Of course, the second data of which the state is determined by the state acquisition function may be stored in the storage unit 20 in order to improve the accuracy of learning or may be used by the second information processing device 30 to be the first data for relearning.

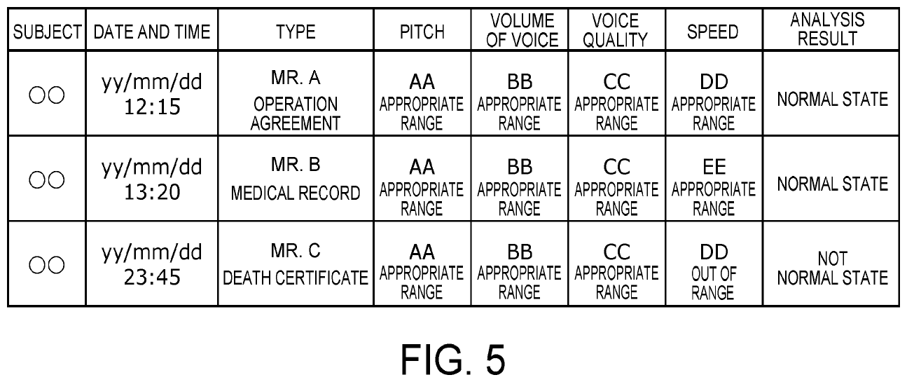

FIG. 5 is a diagram illustrating an example in which results acquired by the state acquisition function are output in time series. For example, different types of clinical information are input for the same subject. By analyzing the voice data, parameters such as a pitch and a volume of a voice are acquired. The state acquisition function compares each of these parameters with a threshold and determines whether the parameter is within a range in a normal state. As described above, individual parameters may be determined, or a combination of several parameters may be used for determination.

It is determined that the parameter is in a normal state for input of an operation agreement of Mr. A, that the parameter is in a normal state for input of the medical record of Mr. B, and that the parameter is not in a normal state for the input of the death certificate of Mr. C. For the input of the clinical information of Mr. A and Mr. B, for example, even if the speed belongs to different ranges, it is determined that the parameters are in a normal state based on the result of semantic analysis of the medical care contents.

On the other hand, for the input of the clinical information of Mr. A and Mr. C, each parameter indicates an equivalent value. However, as a result of performing semantic analysis of the medical care content, it is assumed that the voice speed is outside the appropriate range for the input of the clinical information of Mr. C. In this case, the state acquisition function can determine that the state of the subject is a state different from a normal state at the input timing of the clinical information of Mr. C.

Parameters that are not in a normal state or are not in an appropriate range may be displayed in an easy-to-understand manner by changing colors or changing a display method. A personnel such as a hospital manager or a manager of a subject can grasp the normal difference from the voice input of the subject, thereby quickly detecting the health condition.

(Modified Example)

In FIG. 1, as an example, the first information processing device 10 and the second information processing device 30 are separately provided, but the present invention is not limited thereto. For example, the first information processing device 10 and the second information processing device 30 may be the same information processing apparatus.

As another example, there may be a mode in which the first information processing device 10 only includes the input unit 100, a function for transmitting information acquired from the input unit 100 to the storage unit 20 or the like, and an output function, and the other functions are realized in the second information processing device 30.

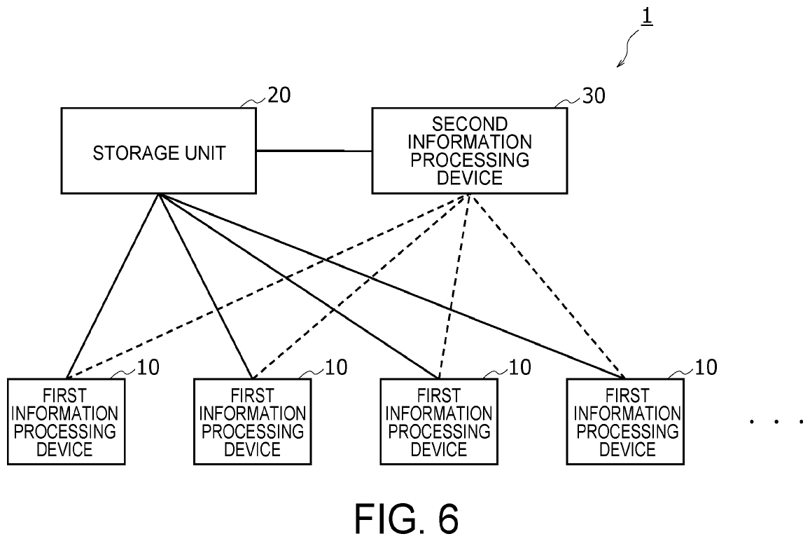

FIG. 6 is an example of the information processing system 1 and is a diagram illustrating the plurality of first information processing devices 10 including at least the input unit 100, and the storage unit 20 and the second information processing device 30 connected to the plurality of first information processing devices 10. As an example, the first information processing device 10 may be a client, and the second information processing device 30 may be installed as a server.

The first information processing device 10 may be provided, for example, for each examination room and each treatment room. The second information processing device 30, which is a server, performs comprehensive database processing and learning processing based on the information acquired from the plurality of first information processing devices 10, which are clients, and the predetermined first information processing device 10 may be a mode of checking the state of the subject by referring to a result of the learning processing and the database.

As illustrated in FIG. 6, each of the first information processing devices 10 may be connected to the storage unit 20 as indicated by a solid line, may be connected to the second information processing device 30 as indicated by a broken line, or may be in a state where these connections are mixed. In addition, a mode may be possible in which an intermediate server is provided, and the first information processing device 10, the storage unit 20, and the second information processing device 30 are appropriately connected via the intermediate server.

With this arrangement, the subject inputs the first data or the second data from a place where one of the first information processing devices 10 exists, and the second information processing device 30 can perform learning by using the data input at a plurality of places. In addition, by rotating the storage unit 20, a mode of acquiring a state of a subject that is not a normal state from the arbitrary first information processing device 10 or acquiring a state of a subject from the first information processing device 10 that cannot access the subject but can be accessed by an administrator is also possible.

The database described in each of the above embodiments may be substituted by using a model of machine learning called a neural network model. In this case, the learning function may use the acquired first data as teacher data and training data to learn a model in each subject by an appropriate arbitrary machine learning method. The state acquisition function and the analysis function can acquire a state of the subject or the scalar or vector related to the state of the subject by inputting at least the voice data of the second data to the learned model learned by the learning function. If necessary, the state acquisition function can determine a normal state or a state different from the normal state by performing threshold processing on the acquired value. The analysis function can process this threshold over time to improve accuracy.

According to at least one embodiment described above, it is possible to improve the accuracy of acquiring the state of the subject, particularly the medical worker, based on the voice information.

In the above description, an example is described in which “processors” in the processing circuitries 102 and 300 read a program corresponding to each processing function from the storage circuit 20 and executes the program, but the embodiment is not limited thereto. The term “processor” means, for example, a circuit such as a central processing unit (CPU), a graphics processing unit (GPU), an application specific integrated circuit (ASIC), or a programmable logic device (for example, a simple programmable logic device (SPLD), a complex programmable logic device (CPLD), and a field programmable gate array (FPGA)). When the processor is, for example, a CPU, the processor reads and executes a program stored in the storage circuit to realize each processing function. Meanwhile, when the processor is, for example, an ASIC, the corresponding processing function is directly incorporated as a logic circuit into a circuit of the processor instead of storing the program in the storage circuit. Note that each processor of the present embodiment is not limited to a case where each processor is configured as a single circuit, and a plurality of independent circuits may be combined to be configured as one processor to realize the processing function. Furthermore, a plurality of components in FIG. 1 and the like may be integrated into one processor to realize a processing function thereof.

Although several embodiments have been described, these embodiments have been presented as examples and are not intended to limit the scope of the invention. These embodiments can be realized in various other modes, and various omissions, substitutions, changes, and combinations of the embodiments can be made without departing from the gist of the invention. These embodiments and modifications thereof are included in the scope and gist of the invention and are included in the invention described in the claims and the equivalent scope thereof.

For the embodiments described above, following notes are discloses as one aspect and alternative features of the invention.

(Note 1) A medical information processing apparatus that stores medical information of a patient, the apparatus comprising a processing circuitry configured to:
perform voice recognition processing on a voice of a user;
input the medical information based on a result of the voice recognition processing;
acquire a mental state of the user by analyzing the voice of the user; and
store an analysis result of a mental state analysis section in a memory.

(Note 2) The voice of the user may be stored in the memory in association with at least one piece of information among:
a place where the user inputs the voice;
a time zone in which the user inputs the voice;
an input amount by which the user inputs the voice;
a device to which the user inputs the voice;
a special input circumstance at a timing when the user inputs the voice; and
information obtained by semantically analyzing an input content of the voice input by the user.

(Note 3) The state of the user may include information of a feeling of the user.

(Note 4) The processing circuitry may further register a result of the learning in a database.

(Note 5) The medical information processing apparatus may further comprise an input unit configured to acquire a second voice of the user who is a medical worker, and the processing circuitry may acquire a state of the user from the second voice based on the database.

(Note 6) The processing circuitry may apply the second voice in the database and may perform threshold processing to acquire whether the state of the user is a state the same as a normal state or a state different from a normal state.

(Note 7) The processing circuitry may further analyze a temporal tendency of the second voice determined to be in the state different from the normal state in the database.

(Note 8) The medical information processing apparatus may further comprises an output unit configured to output information, and the processing circuitry may output, via the output unit, reference information for determining the state of the user from the voice of the user stored in the database together with information indicating a state at a timing when the clinical information is input in the second voice.

(Note 9) A medical information processing system comprising:
a first information processing device having a first processing circuitry configured to acquire, from an acquired voice of a user who is a medical worker, a state of the user based on a database;
a second information processing device having a second processing circuitry configured to
   perform voice recognition processing on the voice of the user,
   input the medical information based on a result of the voice recognition processing,
   acquire a mental state of the user by analyzing the voice of the user, and
   store an analysis result of a mental state analysis section in a memory; and
a storage unit configured to store the database,
wherein the first information processing device acquires the state of the user by using the acquired second data based on the database generated by the second information processing device.

(Note 10) A non-transitory computer readable medium that stores a program causing the processing circuitry to execute:
inputting medical information of a patient by performing voice recognition processing on a voice of a user;
acquiring a mental state of the user by analyzing the voice of the user; and
causing a mental state storage section to store an analysis result of a mental state analysis section in a memory.

(Note 11) A program causing the processing circuitry to execute:
inputting medical information of a patient by performing voice recognition processing on a voice of a user;
acquiring a mental state of the user by analyzing the voice of the user; and
causing a mental state storage section to store an analysis result of a mental state analysis section in a memory.

1. A medical information processing apparatus that stores medical information of a patient, the apparatus comprising a processing circuitry configured to:
perform voice recognition processing on a voice of a user;
input the medical information based on a result of the voice recognition processing;
acquire a mental state of the user by analyzing the voice of the user; and
store an analysis result of a mental state analysis section in a memory.

2. The medical information processing apparatus according to claim 1,
wherein the voice of the user is stored in the memory in association with at least one piece of information among:
a place where the user inputs the voice;
a time zone in which the user inputs the voice;
an input amount by which the user inputs the voice;
a device to which the user inputs the voice;
a special input circumstance at a timing when the user inputs the voice; and
information obtained by semantically analyzing an input content of the voice input by the user.

3. The medical information processing apparatus according to claim 1,
wherein the state of the user includes information of a feeling of the user.

4. The medical information processing apparatus according to claim 1,
wherein the processing circuitry further registers a result of the learning in a database.

5. The medical information processing apparatus according to claim 4, further comprising:
an input unit configured to acquire a second voice of the user who is a medical worker,
wherein the processing circuitry acquires a state of the user from the second voice based on the database.

6. The medical information processing apparatus according to claim 5,
wherein the processing circuitry applies the second voice in the database and performs threshold processing to acquire whether the state of the user is a state the same as a normal state or a state different from a normal state.

7. The medical information processing apparatus according to claim 6,
wherein the processing circuitry further analyzes a temporal tendency of the second voice determined to be in the state different from the normal state in the database.

8. The medical information processing apparatus according to claim 6, further comprising:
an output unit configured to output information,
wherein the processing circuitry outputs, via the output unit, reference information for determining the state of the user from the voice of the user stored in the database together with information indicating a state at a timing when the clinical information is input in the second voice.

9. A medical information processing system comprising:
a first information processing device having a first processing circuitry configured to acquire, from an acquired voice of a user who is a medical worker, a state of the user based on a database;
a second information processing device having a second processing circuitry configured to
perform voice recognition processing on the voice of the user,
input the medical information based on a result of the voice recognition processing,
acquire a mental state of the user by analyzing the voice of the user, and
store an analysis result of a mental state analysis section in a memory; and
a storage unit configured to store the database,
wherein the first information processing device acquires the state of the user by using the acquired second data based on the database generated by the second information processing device.

10. A non-transitory computer readable medium that stores a program causing the processing circuitry to execute:
inputting medical information of a patient by performing voice recognition processing on a voice of a user;
acquiring a mental state of the user by analyzing the voice of the user; and
causing a mental state storage section to store an analysis result of a mental state analysis section in a memory.
